(19) United States
(12) Patent Application Publication
(10) Pub. No.: US 2009/0253157 A1
(43) Pub. Date: Oct. 8, 2009

(54) METHOD OF DIRECTED DIFFERENTIATION OF PORCINE EMBRYONIC STEM CELLS AND USING THE SAID CELLS IN DRUG SCREENING

(75) Inventors: Jenn-Rong Yang, Kaohsiung City (TW); Lih-Ren Chen, Tainan City (TW); Yow-Ling Shiue, Tainan City (TW); Chia-Hsin Liao, Ji-an Township (TW)

Correspondence Address: OCCHIUTI ROHLICEK & TSAO, LLP, 10 FAWCETT STREET, CAMBRIDGE, MA, 02138, US

(73) Assignee: Livestock Research Institute, Council of Agriculture, Executive Yuan, Sinhua Township (TW)

(21) Appl. No.: 12/098,592

(22) Filed: Apr. 7, 2008

(51) Int. Cl. C12Q 1/48; C12N 5/06; C12Q 1/34; C12Q 1/02

(52) U.S. Cl. 435/15; 435/325; 435/377; 435/29; 435/18

(57) ABSTRACT

The present invention relates to a method of directed differentiation of porcine embryonic stem cells into specific neural lineages. The present invention also relates to a method for identifying neurogenic stimulator using the said porcine embryonic stem cells.

### BACKGROUND OF THE INVENTION

Embryonic stem (ES) cells are able to self-renew, and proliferate continuously in vitro with the undifferentiated characteristics (Evans and Kaufman, 1981 Nature 292: 154-156; Martin, 1981 Proc. Natl. Acad. Sci. USA. 78: 7634-7638). In responding to suitable conditions, they can be induced to differentiate into cells of all three primary germ layers. There were many remarkable results in directing the human ES (hES) cell differentiation into neuronal cells (Thomson et al., 1998 Science 282: 1145-1147; Reubinoff et al., 2001 Nat. Biotechnol. 19: 1134-1140; Schuldiner et al., 2001 Brain Res. 913: 201-205.; Wichterle et al., 2002 Cell. 2002. 110: 385-397), pancreatic β cells (Assady et al., 2001 Diabetes 50: 1691-1697; Moritoh et al., 2003 Diabetes. 2003. 52: 1163-1168.), and cardiomyocytes (Kehat et al., 2001 J. Clin. Invest. 108: 407-414). These results reveal a potential of the clinical application of hES cells in the therapy of diseases such as Parkinson's disease, spinal cod injure, diabetes, and heart diseases.

Over the past few years, mouse embryonic stem (mES) has been established as an animal model in fundamental stem cell research. However, recent studies have further underlined the existence of difference between human and mouse embryonic carcinoma (EC) and embryonic stem (ES) cells, indicating that data from the laboratory mouse cannot necessarily be extrapolated to human development, and that study of both human and murine development are complementary. Thus, using animals other than mice as animal models in such researches is a need.

Although the establishment of pluripotent ES cell lines from domestic species is much more difficult than that in murine species, porcine ES (pES) cell lines have been successfully derived from inner cell mass of the blastocysts (Chen et al., 1999 Theriogenology 52: 195-212; Li et al., 2003, Mol. Reprod. Dev. 65: 429-434). The pES cells were very similar to hES cells in many characteristics, including colony morphology, feeder-dependent and refractory to leukemia inhibitory factor (LIF) in culture, and expression of stage-specific embryonic antigen 3/4 (SSEA 3/4) but not the SSEA 1, which is characterized only to murine ES cells. Therefore, pES cells might serve as an excellent model in the study and development of regenerative medicine in human. Domestic swine are demonstrated very similar to human in anatomic, immunologic, and physiologic characteristics; and the sizes of their organs are fairly comparable to those of human (Phillips and Tumbleson, 1986 Models in ‘swine in biomedical research’, Vol. 1. Tumbleson M. E., editor. New York: Plenum Press. pp. 437-440.; Prelle et al., 1999 Cells Tissues Organs 165: 220-236.). Moreover, swine have been demonstrated as excellent animal models in therapeutic development for various human diseases, including congenital heart disease (Swindle et al., 1992 Iowa: Iowa State University Press. pp. 176-184), hypertension (Zambraski et al., 1992 Iowa: Iowa State University Press. pp. 290-301), organ transplantation (Hall et al., 1986 In: Swine in Biomedical Research, M. E. Tumbleson, ed. New York: Plenum Press. pp. 373-376.; Flye, 1992 Iowa: Iowa State University Press. pp. 44-56), pharmacology and toxicology (Kurihara-Bergstrom et al., 1986 Lab. Anim. Sci. 36: 396-399; Feletou and Teisseire, 1992 Iowa: Iowa State University Press. pp. 74-95). Taken together, the study of pES cells could provide information that is not possible to obtain from human stem cell researches. Therefore, an improved technique to induce differentiation of pES cells is a need.

### BRIEF SUMMARY OF THE INVENTION

The main object of the present invention is to provide a cell system for producing neurons via directed differentiation comprising a porcine embryonic stem (pES) cell line derived from pre-implantation blastocytes. More specifically, the ES cell line is the M215-3 strain.

The other object of the present invention is to provide a method for inducing neural differentiation of ES cells in a two-stage protocol comprising: (1) culturing ES cells in a suspension culture in ES-cell culture medium (ESM) containing a neurogenic stimulator for a 12-day period; (2) collecting the cultured cells from the ESM; (3) plating the collected cells onto gelatin-coated dishes and culturing them for an appropriate period to allow differentiation into dopaminergic neurons, cholinergic (ChAT) neurons or GABAergic (GABA) neurons.

The another object of the present invention is to provide a method for identifying a neurogenic stimulator comprising the steps of: (1) providing pES cells of the M215-3 strain; (2) culturing the cells in a suspension culture in ES-cell culture medium (ESM) containing a potential neurogenic stimulator for a 12-day period; (3) collecting the cultured cells from the ESM; (4) plating the collected cells onto gelatin-coated dishes and culturing them for an appropriate period to allow differentiation into dopaminergic neurons, cholinergic (ChAT) neurons or GABAergic (GABA) neurons; (5) detecting biomarkers of interest expressed by pES cell-derived neural cells to determine the effect of the said potential neurogenic stimulator.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A is a photograph showing the undifferentiated pES cells according to one embodiment of the invention;

FIG. 1B is a photograph showing the in vitro neural differentiation of pES cells according to one embodiment of the invention;

FIG. 1C is a photograph showing the neural-like network in the high-density replated culture at day 4 after replating according to one embodiment of the present invention;

FIG. 1D is a photograph showing the neural-like network expressing nestin according to one embodiment of the present invention;

FIG. 2A is a photograph showing the bipolar-like neuron in the low-density culture at day 6 according to one embodiment of the present invention;

FIG. 2B is a photograph showing the multipolar-like neuron in the low-density culture at day 20 according to one embodiment of the present invention;

FIG. 2C is a photograph showing the astrocyte-like neuron at day 6 according to one embodiment of the present invention;

FIG. 2D is a photograph showing the astrocyte-like neuron at day 20 according to one embodiment of the present invention;

FIG. 3A is a photograph showing a differentiated pES cell expressing neuronal specific marker nestin after immunocytochemical staining according to one embodiment of the present invention. The left figure was photographed under optical microscopy, and the right figure was photographed under fluorescence microscopy;

FIG. 3B is a photograph showing a differentiated pES cell expressing neuronal specific marker NFL after immunocytochemical staining according to one embodiment of the present invention. The left figure was photographed under optical microscopy, and the right figure was photographed under fluorescence microscopy;

FIG. 3C is a photograph showing a differentiated pES cell expressing neuronal specific marker MAP2 after immunocytochemical staining according to one embodiment of the present invention. The left figure was photographed under optical microscopy, and the right figure was photographed under fluorescence microscopy;

FIG. 3D is a photograph showing differentiated pES cells expressing neuronal specific marker TH after immunocytochemical staining according to one embodiment of the present invention. The left figure was photographed under optical microscopy, and the right figure was photographed under fluorescence microscopy; and

FIG. 3E is a photograph showing differentiated pES cells expressing neuronal specific marker ChAT after immunocytochemical staining according to one embodiment of the present invention. The left figure was photographed under optical microscopy, and the right figure was photographed under fluorescence microscopy.

### DETAILED DESCRIPTION OF THE INVENTION

As used herein the following terms may be used for better interpretation of claims and specification.

The articles “a” and “an” are used herein to refer to one or more than one (i.e., at least one) of the grammatical object of the article. By way of example, “an element” means one element or more than one element.

The main object of the present invention relates to a cell system for producing neurons via directed differentiation comprising a porcine embryonic stem (pES) cell line derived from pre-implantation blastocytes. More specifically, the pES cell line is the M215-3 strain. As used herein, the term “stem cell line” refers to an inbred stem cell that maintains the parents' properties after subculture. The pES cell line of M215-3 strain comprises an exogenous reporter gene of the green fluorescent protein. The reporter protein allows tracing cell migration or evaluating co-introduced exogenous genes more conveniently.

The term “porcine embryonic stem cell” used herein refers to pluripotent porcine embryonic stem cells derived from the inner cell mass of blastocysts. In one embodiment, the blastocyst is obtained from the species Taiwan Livestock Research Institute Black Pig No. 1.

The present invention also provides a method for inducing neural differentiation of ES cells in a two-stage protocol comprising: (1) culturing ES cells in a suspension culture in ES-cell culture medium (ESM) containing a neurogenic stimulator for a 12-day period; (2) collecting the cultured cells from the ESM; (3) plating the collected cells onto gelatin-coated dishes and culturing them for an appropriate period to allow differentiation into dopaminergic neurons, cholinergic (ChAT) neurons or GABAergic (GABA) neurons.

Any ordinary neurogenic stimulator can be used to induce the neural differentiation into an interested subtype in the two-stage protocol of the present invention. In accordance with one embodiment of the present invention, the neurogenic stimulator is selected from the group consisting of: (1) retinoic acid (RA); (2) sonic hedgehog (SHH); (3) fibroblast growth factor (FGF); and combinations thereof. Preferably, the concentration of retinoic acid (RA) in conditioned media can be 1 μM, the concentration of sonic hedgehog (SHH) in conditioned media can be 200 ng/ml, and the concentration of fibroblast growth factor (FGF) in conditioned media can be 100 ng/ml.

The present invention also provides a method for identifying a neurogenic stimulator comprising the steps of: (1) providing pES cells of the M215-3 strain; (2) culturing the cells in a suspension culture in ES-cell culture medium (ESM) containing a potential neurogenic stimulator for a 12-day period; (3) collecting the cultured cells from the ESM; (4) plating the collected cells onto gelatin-coated dishes and culturing them for an appropriate period to allow differentiation into dopaminergic neurons, cholinergic (ChAT) neurons or GABAergic (GABA) neurons; (5) detecting biomarkers of interest expressed by pES cell-derived neural cells to determine the effect of the said potential neurogenic stimulator.

The term “biomarker” used herein refers to specific antigens present on cell surface or specific proteins produced by the cells when differentiating into specific lineages. One skilled in the art would know how to choose a correct biomarker to detect the interested neuron. The detection can be conducted with any ordinary biological or chemical method, for example, ELISA or immunocytochemical staining. In one embodiment, the biomarkers are selected from the group consisting of nestin, neurofilament protein (NFL), microtubule associated protein 2 (MAP2), glial fibrillary acidic protein (GFAP), S100 protein, A2B5, cyclic nucleotide phosphohydrolase (CNPase), O4, tyrosine hydroxylase (TH), dopamine transporter (DAT), choline acetyltransferase (ChAT) and Gamma-aminobutyric acid (GABA).

The invention is described more specifically with the following embodiments. It should be noted that the following descriptions of preferred embodiments of this invention are presented herein for the purpose of illustration and description only; it is not intended to be exhaustive or to be limited to the precise form disclosed.

### EXAMPLE 1

### Preparation of Porcine Embryonic Stem Cell Culture

The pES cells of the M215-3 strain derived from the pre-implantation blastocysts of Taiwan Livestock Research Institute Black Pig No. 1 (a topcrossing breed established from Taoyuan and Duroc pigs) were maintained in ES-cell culture medium (ESM) consisting of Dulbecco's modified eagle medium (DMEM, high glucose and no pyruvate, Invitrogen, Grand Island, N.Y., USA) supplemented with 1 mM L-glutamine (Sigma-Aldrich, St. Louis, Mo., USA), 0.1 mM β-2-mercaptoethanol (Sigma-Aldrich), 10 mM MEM non-essential amino acids (Sigma-Aldrich), 0.03 mM adenosine (Sigma-Aldrich), 0.03 mM guanosine (Sigma-Aldrich), 0.03 mM cytidine (Sigma-Aldrich), 0.03 mM uridine (Sigma-Aldrich), 0.01 mM thymidine (Sigma-Aldrich), antibiotics (50 units/ml penicillin G and 50 μg/ml streptomycin sulfate, Invitrogen) and 16% fetal bovine serum (FBS, Invitrogen) as described previously (Chen et al., 1991, J. Chin. Soc. Anim. Sci. 20: 326-339). Briefly, the pluripotent pES cells were propagated on a feeder layer of mitomycin C (Sigma-Aldrich) inactivated STO cells (mouse embryonic fibroblasts, CRL-1503, USA) in gelatin-coated Multidish 4 Wells® (Nunc 176740, Roskilde, Denmark) in ESM at 39° C. with an atmosphere of 5% CO₂ in air. For the maintenance of undifferentiation, pES colonies were regulatory subcultured with 0.25% trypsin-0.02 mM EDTA (Invitrogen) and re-plated onto fresh STO feeder layers every 5 to 7 days.

### EXAMPLE 2

### In Vitro Induction of Neural Differentiation

Confluent pES cells were trypsinized and subjected to a two-stage in vitro induction protocol for neural differentiation. They were firstly taken into a suspension culture in an Easy Flask® (NUNC 169900, Roskilde, Denmark) for a 12-day period of neural induction. Neural induction is initiated by plating undifferentiated pES cells as single-cell suspension at a number of 1×10⁵ cells totally in ESM medium withdrawing FBS and containing 1 μM retinoic acid (RA, Sigma-Aldrich), 200 ng/ml sonic hedgehog (SHH, R&D systems, Minneapolis, Minn., USA) and 100 ng/ml fibroblast growth factor (FGF, Sigma-Aldrich) as described by Barberi et al. (Nat. Biotechnol. 2003. 21: 1200-1207). Five combined treatments of neurogenic stimulators for neural induction were applied: (1) RA, (2) RA+SHH, (3) RA+FGF, (4) SHH+FGF, (5) RA+SHH+FGF. After 12 days of induction, the pES cell-derived neural cells were plated onto gelatin-coated 4-well dishes in high-density (2×10⁴ cells/ml) or low-density (5×10³ cells/ml). These cells were then cultured in DMEM/F12 medium (Sigma-Aldrich) supplemented with 10% FBS, 20 ng/ml human recombinant epidermal growth factor (hrEGF, Invitrogen), 20 ng/ml human recombinant basic fibroblast growth factor (hrbFGF, Invitrogen), and 1:100 N2 (Invitrogen), and allowed for the propagation of neural precursor cells (Pan et al., 2005 Biochem. Biophys. Res. Commun. 327: 548-556). After plating onto the 4-well dish, the induced pES cells were treated with trypsin/EDTA and collected at days 2, 4, 6, 8, 10, and 12, respectively, to analyze the neural-related development by immunocytochemical staining.

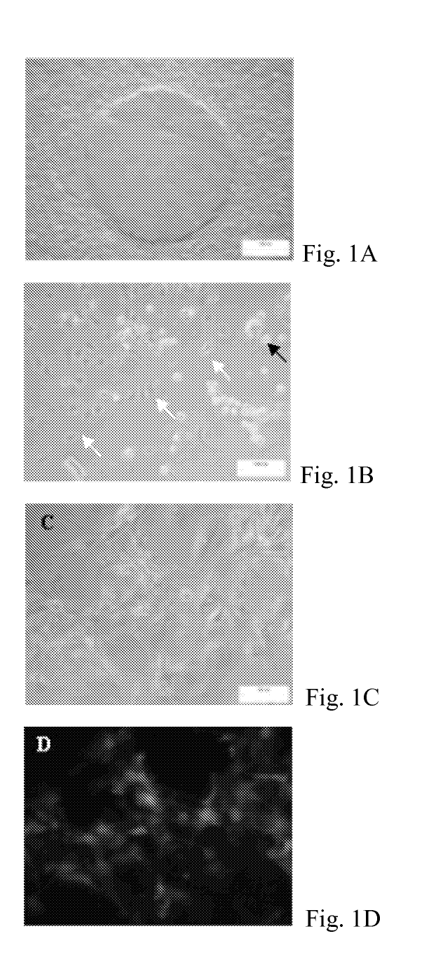

Referring to FIG. 1A, the colony of pES cells derived from the pre-implantation blastocyst and co-cultured in mitomycin C-inactived STO feeder layer maintained typical undifferentiated morphology. The undifferentiated pES colonies formed compact circular colonies in STO feeder layer culture. They had indistinct cell membranes between pES cells but had distinct cell border between STO cells.

Referring to FIG. 1B, the neural differentiation of pES cells was induced by 1 μM RA, 200 ng/ml SHH, and 100 ng/ml FGF in vitro. Throughout 12 days of suspension culture and then re-plating onto gelatin-coated 4-well dishes, the unipolar neuron with a cell body-like (black arrowhead) and axon-like (white arrowheads) structure was observed at day 4 after replating.

Referring to FIG. 1C, a neural-like network structure has been observed in the high-density replated culture (2×10⁴ cells/ml) at day 4 after replating. And referring to FIG. 1D, the nestin was expressed by the neural-like network shown previously in FIG. 1C.

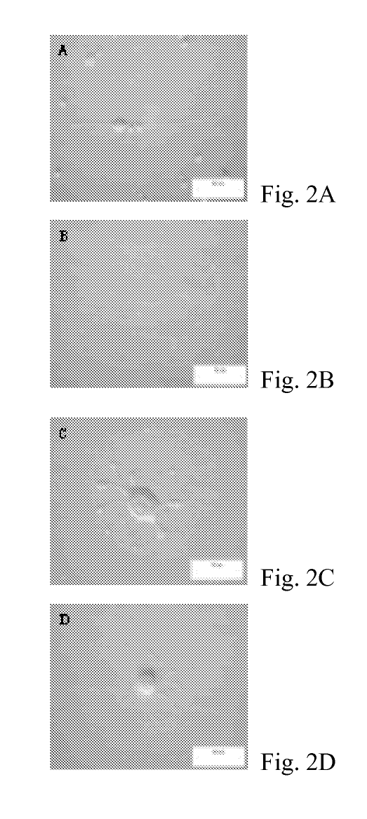

Referring to FIG. 2A, when replated in the low-density culture (5×10³ cells/ml), the bipolar-like neuron was observed at day 6. Referring to FIG. 2B, the multipolar-like neuron was developed at day 20. FIG. 2C, the astrocyte-like neuron was observed when replated at day 6, and referring FIG. 2D, the astrocyte-like neuron was observed when replated at day 20.

### EXAMPLE 3

### Immunocytochemical Staining

Cells for immunocytochemical analysis were fixed in 10% formalin at room temperature for 30 minutes. The cells were washed with phosphate buffered saline (PBS, Sigma-Aldrich), then incubated with 0.3% Triton X-100 (Sigma-Aldrich) for 10 minutes, 3% H₂O₂ (Sigma-Aldrich) for 5 minutes and incubated in blocking buffer (5% FBS) at room temperature for 2 hours. After being washed with PBS, the cells were incubated overnight with appropriate dilution of primary antibody, or combination of antibodies at 4° C. (Li et al., 2005 Nat. Biotechnol. 2005. 23: 215-221). Subsequently, cells were incubated with secondary antibody of Fluorescein (FITC)-conjugated AffiniPure Goat Anti-Rabbit IgG, Goat Anti-Mouse IgG, IgM (1:200, Jackson Immunoresearch Laboratories, West Baltimore Pike, Pa., USA) for a minimum of 2 hours, followed by washing in PBS.

Primary antibodies for neural specific binding included nestin, neurofilament protein 68 kDa (NFL, 1:200), microtubule associated protein 2 (MAP2), glial fibrillary acidic protein (GFAP), S100 protein, A2B5, cyclic nucleotide phosphohydrolase (CNPase), O4, tyrosine hydroxylase (TH), dopamine transporter (DAT), choline acetyltransferase (ChAT) and GABA. All primary antibodies are from Chemicon (Temecula, Calif., USA) and hybridized in a 200 folds of dilution. Fluorescent cells were visualized using an inverted fluorescent microscopy (DM IRB, Leica, Wetzlar, Germany) equipped with digital camera (E995, Nikon, Japan). Negative controls for each fluorophore-conjugated secondary antibody, carried out without the addition of the primary antibody, exhibited no non-specific binding of the secondary antibodies.

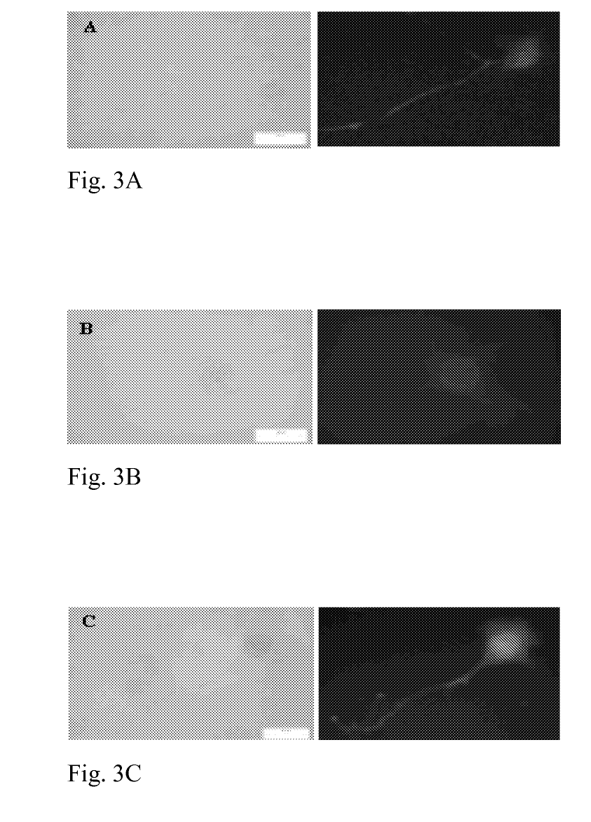

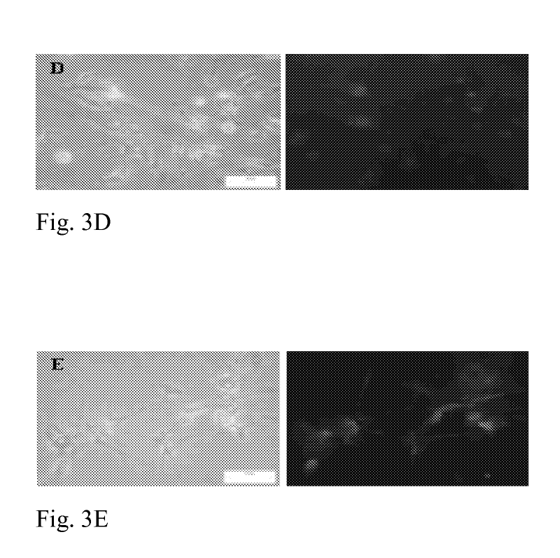

Referring to FIG. 3A, nestin positive cells were derived from pES cells by all of the combined treatments at day 4 after re-plating. Referring to FIG. 3B, at day 6 after replating, differentiated cells expressed neuronal specific marker neurofilament protein (NFL). Referring to FIG. 3C, at day 6 after replating, differentiated cells expressed neuronal specific marker microtubule associated protein 2 (MAP2). Referring to FIG. 3D, differentiated cells expressed neuronal specific marker tyrosine hydroxylase (TH) after combined treatment. Referring to FIG. 3E, differentiated cells expressed neuronal specific marker choline acetyltransferase (CHAT) after combined treatment. Taken together, these results of immunocytochemical staining showed that the pES cells induced by RA, SHH and FGF can differentiate into specific subtypes of neurons.

### EXAMPLE 4

### Efficiency of pES Cells Differentiating into Neural Lineages

The directed differentiation of pES cells into neural lineages was monitored and lineage potential of pES cells was determined. The results of the immunocytochemical analysis described in Example 3 were summarized in the following tables. The efficiency of pES cells differentiating into neural lineages was presented as mean±S.D. All results were derived from at least three independent experiments. Data were analyzed by analysis of variance using the General Linear Model (GLM) procedure of SAS (SAS Institute, 1996). Differences between means were deemed as significant when p<0.01.

TABLE 1
The percentage of pES cell-derived neural lineages expressing nestin from
day 2 to day 12 after the RA, SHH and FGF combined treatments

| | Treatments | | | | |
|---|---|---|---|---|---|
| Nestin | RA | RA + SHH | RA + FGF | SHH + FGF | RA + SHH + FGF |
| D2 | 86.2 ± 7.9^{ab} | 92.7 ± 3.2^{a} | 90.2 ± 6.4^{a} | 87.7 ± 8.5^{a} | 88.7 ± 5.9^{a} |
| D4 | 90.5 ± 4.7^{a} | 89.1 ± 4.8^{a} | 84.9 ± 6.8^{b} | 82.3 ± 8.3^{ab} | 80.3 ± 6.8^{b} |
| D6 | 87.8 ± 4.3^{ab} | 75.6 ± 10.9^{b} | 83.5 ± 7.0^{b} | 81.9 ± 9.4^{ab} | 82.5 ± 3.5^{b} |
| D8 | 81.2 ± 8.0^{bc} | 77.9 ± 5.7^{b} | 75.4 ± 8.8^{c} | 79.3 ± 10.7^{b} | 73.5 ± 5.4^{c} |
| D10 | 82.4 ± 11.4^{bc} | 73.8 ± 7.0^{b} | 75.3 ± 6.1^{c} | 65.2 ± 4.5^{c} | 73.7 ± 7.2^{c} |
| D12 | 77.9 ± 6.6^{c} | 74.6 ± 4.5^{b} | 77.2 ± 4.8^{c} | 62.3 ± 3.8^{c} | 78.8 ± 6.6^{bc} |

^{a,b,c}Different superscripts in the same column are significantly different (p < 0.01).

TABLE 2
The percentage of pES cell-derived neural lineages expressing NFL from
day 2 to day 12 after the RA, SHH and FGF combined treatments.

| | Treatments | | | | |
|---|---|---|---|---|---|
| NFL | RA | RA + SHH | RA + FGF | SHH + FGF | RA + SHH + FGF |
| D2 | 93.9 ± 3.9^{a} | 89.4 ± 4.4^{a} | 91.7 ± 6.2^{a} | 89.8 ± 8.6^{a} | 87.1 ± 5.0^{a} |
| D4 | 90.3 ± 4.8^{ab} | 83.9 ± 4.3^{b} | 84.6 ± 6.1^{b} | 81.2 ± 7.1^{bc} | 84.0 ± 7.2^{ab} |
| D6 | 91.8 ± 6.0^{a} | 81.8 ± 6.2^{b} | 80.3 ± 8.4^{bc} | 82.0 ± 7.7^{bc} | 81.5 ± 5.0^{bc} |
| D8 | 85.8 ± 4.2^{b} | 81.7 ± 5.1^{b} | 74.7 ± 10.0^{d} | 84.3 ± 8.8^{ab} | 83.6 ± 6.1^{ab} |
| D10 | 84.4 ± 7.7^{b} | 83.2 ± 3.7^{b} | 78.4 ± 5.5^{cd} | 81.0 ± 10.4^{bc} | 83.1 ± 6.0^{abc} |
| D12 | 74.4 ± 12.3^{c} | 81.0 ± 9.2^{b} | 81.1 ± 3.5^{bc} | 75.9 ± 9.0^{c} | 78.4 ± 4.1^{c} |

^{a,b,c,d}Different superscripts in the same column are significantly different (p < 0.01).

Referring to Table 1, the pES cells were under directed differentiation by 5 combined treatments, followed by neural specific immunocytochemical staining with nestin. During the 12-day directed differentiation, high level of nestin expression by pES was observed at day 2. However, with the increase of culture days, the expression of nestin decreased, suggesting that the cells expressing neural precursors decreased and the said cells differentiated toward other types of neural cells. Referring to Table 2, during the same culture procedure after induction, NFL positive cells also show the same trend.

TABLE 3
The percentage of pES cell-derived neural lineages expressing GFAP from
day 2 to day 12 after the RA, SHH and FGF combined treatments.

| | Treatments | | | | |
|---|---|---|---|---|---|
| GFAP | RA | RA + SHH | RA + FGF | SHH + FGF | RA + SHH + FGF |
| D2 | 9.8 ± 3.8 | 72.0 ± 12.3^{a} | 24.5 ± 10.7^{c} | 22.8 ± 10.1^{b} | 51.5 ± 14.5^{b} |
| D4 | 7.6 ± 4.2 | 58.6 ± 15.5^{b} | 25.1 ± 12.5^{c} | 29.5 ± 15.2^{ab} | 53.1 ± 11.7^{b} |
| D6 | 7.7 ± 3.6 | 59.4 ± 10.1^{b} | 55.0 ± 11.4^{a} | 28.8 ± 14.5^{ab} | 71.1 ± 7.8^{a} |
| D8 | 7.3 ± 1.5 | 55.0 ± 10.1^{b} | 37.0 ± 13.7^{b} | 36.4 ± 15.7^{a} | 63.2 ± 8.8^{a} |
| D10 | 7.7 ± 4.5 | 57.5 ± 6.9^{b} | 19.0 ± 8.6^{cd} | 36.0 ± 12.4^{a} | 45.0 ± 12.1^{b} |
| D12 | 7.6 ± 2.3 | 57.1 ± 5.7^{b} | 11.6 ± 8.1^{d} | 21.5 ± 6.8^{b} | 28.0 ± 13.6^{c} |

^{a,b,c}Different superscripts in the same column are significantly different (p < 0.01).

Referring to Table 3, the pES cells were treated with RA and other different combined treatments to see astrocytic differentiation. Less than 10% of the differentiated cells induced by RA only showed to be GFAP positive after culture. The number of cells expressing GFAP after RA treatment is apparently less than those after other combined treatments. On the contrary, cells treated with RA+SHH and RA+SHH+FGF had the best results, both exhibiting percentages over 50% at most time during the 12-day post-treatment period.

TABLE 4
The percentage of pES cell-derived neural lineages expressinf A2B5 from
day 2 to day 12 after the RA, SHH and FGF combined treatments.

| | Treatments | | | | |
|---|---|---|---|---|---|
| A2B5 | RA | RA + SHH | RA + FGF | SHH + FGF | RA + SHH + FGF |
| D2 | 83.7 ± 12.6 | 89.0 ± 4.0^{a} | 88.9 ± 5.4^{a} | 74.4 ± 7.3^{b} | 82.4 ± 5.1^{a} |
| D4 | 86.6 ± 3.3 | 85.3 ± 11.0^{ab} | 84.4 ± 7.0^{b} | 76.0 ± 8.6^{b} | 80.5 ± 5.8^{ab} |
| D6 | 87.6 ± 7.4 | 76.9 ± 5.7^{bc} | 82.2 ± 7.1^{bc} | 82.1 ± 9.4^{a} | 82.3 ± 5.6^{a} |
| D8 | 86.9 ± 9.3 | 71.5 ± 5.9^{cd} | 78.5 ± 6.1^{c} | 83.9 ± 8.4^{a} | 81.6 ± 4.8^{ab} |
| D10 | 86.3 ± 1.9 | 72.3 ± 9.1^{cd} | 79.3 ± 5.7^{c} | 76.7 ± 5.7^{b} | 77.2 ± 6.8^{b} |
| D12 | 84.8 ± 5.2 | 63.7 ± 10.7^{d} | 73.7 ± 11.1^{c} | 71.9 ± 7.1^{b} | 79.5 ± 6.5^{ab} |

^{a,b,c,d}Different superscripts in the same column are significantly different (p < 0.01).

Referring to Table 4, the pES cells were treated with RA and other different combined treatments to see oligodendrocytic differentiation. The ratio of cells expressing A2B5 after RA treatment during the following culture is all above 80%. However, the ratio of cells expressing A2B5 after RA+SHH, RA+FGF and RA+SHH+FGF treatment decreased along with the following culture. In addition, at the SHH+FGF group, the percentage of cells expressing A2B5 showed a high level during day 6 to day 8. This result is different from pES cells treated with other combined treatments in which the percentage decreased with time.

TABLE 5
The percentage of pES cell-derived neural lineages expressing TH from day
2 to day 12 after the RA, SHH and FGF combined treatments

| | Treatments | | | | |
|---|---|---|---|---|---|
| TH | RA | RA + SHH | RA + FGF | SHH + FGF | RA + SHH + FGF |
| D2 | 7.8 ± 2.5 | 87.8 ± 7.5 | 85.7 ± 9.2 | 76.3 ± 11.6^{bc} | 81.1 ± 6.3 |
| D4 | 8.0 ± 4.9 | 86.6 ± 6.4 | 84.8 ± 8.3 | 74.2 ± 10.1^{c} | 81.1 ± 7.0 |
| D6 | 8.7 ± 1.7 | 87.2 ± 10.5 | 86.5 ± 8.1 | 82.2 ± 9.8^{ab} | 82.4 ± 7.5 |
| D8 | 8.5 ± 2.7 | 86.3 ± 6.3 | 88.4 ± 5.7 | 86.8 ± 6.3^{a} | 82.8 ± 5.0 |
| D10 | 8.5 ± 4.2 | 81.7 ± 7.1 | 89.1 ± 5.7 | 82.2 ± 7.7^{abc} | 80.7 ± 4.5 |
| D12 | 8.4 ± 3.5 | 80.2 ± 6.0 | 88.6 ± 5.3 | 74.6 ± 10.1^{bc} | 80.4 ± 5.7 |

^{a,b,c}Different superscripts in the same column are significantly different (p < 0.01).

TABLE 6
The percentage of pES cell-derived neural lineages expressing ChAT from
day 2 to day 12 after the RA, SHH and FGF combined treatments.

| | Treatments | | | | |
|---|---|---|---|---|---|
| ChAT | RA | RA + SHH | RA + FGF | SHH + FGF | RA + SHH + FGF |
| D2 | 87.1 ± 5.6^{ab} | 85.4 ± 7.6^{ab} | 88.6 ± 4.5^{a} | 77.9 ± 12.7^{b} | 77.9 ± 7.2^{b} |
| D4 | 85.8 ± 3.2^{bc} | 84.1 ± 5.0^{b} | 87.9 ± 5.9^{a} | 77.4 ± 9.0^{b} | 84.3 ± 7.3^{a} |
| D6 | 91.8 ± 8.3^{a} | 93.9 ± 3.5^{a} | 85.9 ± 4.7^{ab} | 80.2 ± 8.4^{b} | 83.1 ± 4.7^{ab} |
| D8 | 86.9 ± 4.4^{ab} | 82.9 ± 6.6^{b} | 83.8 ± 5.8^{bc} | 84.9 ± 6.7^{a} | 77.8 ± 4.4^{b} |
| D10 | 89.9 ± 7.2^{ab} | 80.7 ± 8.3^{b} | 81.5 ± 3.7^{c} | 83.3 ± 5.8^{ab} | 80.5 ± 4.4^{ab} |
| D12 | 80.3 ± 6.0^{c} | 80.4 ± 5.9^{b} | 84.0 ± 5.7^{bc} | 80.5 ± 6.5^{ab} | 81.9 ± 6.5^{ab} |

^{a,b,c}Different superscripts in the same column are significantly different (p < 0.01).

Referring to Table 5 and Table 6, the pES cells were treated with RA and four other different combined treatments to see the differentiation of functional neuron. Two specific antigens, TH and ChAT, were chosen to perform immunocellular analysis to understand the ability of pES cells to differentiate into dopaminergic and cholinergic neurons after directed differentiation. For dopaminergic neurons, merely below 10% of the derivative cells expressed TH after pES cells were treated with RA for directed differentiation, suggesting that the RA-only treatment can not lead to better differentiations of dopaminergic neurons. However, after RA+SHH, RA+FGF, and RA+SHH+FGF treatments, the percentage of cells expressing TH during the culture increased by 80% without significant changes. Further, the percentage of cells expressing TH is up to 70% at the beginning in the RA+SHH treatment group. This percentage increased at day 6 and day 8 but decreased in the following days. As to cholinergic neurons, the percentages of cells expressing CHAT in different treatments are all about 80%, suggesting that this kind of directed differentiation is a proper way to obtain high level of cholinergic neurons. This is the most effective way to induce differentiation.

TABLE 7
The percentage of pES cell-derived neural lineages expressing the
biomarkers of neural precursors (nestin) and neuronal (NFL and MAP2), astrocytic
(GFAP and S100), oligodendrocytic (A2B5, CNPase and O4), dopaminergic (TH and
DAT), cholinergic (ChAT) and GABAergic (GABA) neurons at day 6 after the RA,
SHH and FGF combined treatments.

| | Treatments | | | | |
|---|---|---|---|---|---|
| | RA | RA + SHH | RA + FGF | SHH + FGF | RA + SHH + FGF |
| Nestin | 87.8 ± 4.3^{a} | 75.6 ± 10.9^{b} | 83.5 ± 7.0^{a} | 81.9 ± 9.4^{ab} | 82.5 ± 3.5^{ab} |
| NFL | 91.8 ± 6.0^{a} | 81.8 ± 6.2^{b} | 80.3 ± 8.4^{b} | 82.0 ± 7.7^{b} | 81.5 ± 5.0^{b} |
| MAP2 | 86.1 ± 3.2^{a} | 93.9 ± 2.5^{b} | 93.7 ± 3.4^{b} | 83.4 ± 5.1^{a} | 87.4 ± 2.9^{a} |
| GFAP | 7.7 ± 3.6^{d} | 59.4 ± 10.1^{b} | 55.0 ± 11.4^{b} | 28.8 ± 14.5^{c} | 71.1 ± 7.8^{a} |
| S100 | 71.4 ± 12.5^{a} | 49.2 ± 17.4^{c} | 77.3 ± 11.5^{a} | 55.4 ± 11.7^{bc} | 67.0 ± 15.4^{ab} |
| A2B5 | 87.6 ± 7.4^{a} | 76.9 ± 5.7^{b} | 82.2 ± 7.1^{ab} | 82.1 ± 9.4^{ab} | 82.3 ± 5.6^{ab} |
| CNPase | 89.8 ± 4.7^{ab} | 93.8 ± 3.2^{a} | 92.6 ± 3.6^{a} | 88.1 ± 6.1^{ab} | 87.5 ± 9.7^{b} |
| O4 | 90.7 ± 5.9 | 93.3 ± 3.4 | 90.6 ± 4.6 | 89.2 ± 4.2 | 89.4 ± 5.9 |
| TH | 8.7 ± 1.7^{b} | 87.2 ± 10.5^{a} | 86.5 ± 8.1^{a} | 82.2 ± 9.8^{a} | 82.4 ± 75^{a} |
| DAT | 87.3 ± 6.5^{a} | 83.0 ± 9.3^{ab} | 88.6 ± 5.9^{a} | 83.0 ± 9.1^{ab} | 80.6 ± 8.5^{b} |
| ChAT | 91.8 ± 8.3^{a} | 93.9 ± 3.5^{a} | 85.9 ± 4.7^{b} | 80.2 ± 8.4^{c} | 83.1 ± 4.7^{bc} |
| GABA | 80.5 ± 9.2^{a} | 60.9 ± 9.7^{bc} | 57.2 ± 13.0^{c} | 50.9 ± 15.4^{c} | 69.2 ± 13.8^{ab} |

^{a,b,c}Different superscripts in the same row are significantly different (p < 0.01).

Referring to Table 7, pES cells were treated with five different combined treatments. After directed differentiation, the cells are analyzed by different neural specific antigens to investigate the efficiency of differentiation induced by these different combined treatments. The percentage of cells expressing nestin is higher in the RA and RA+FGF groups than in other combined treatment groups. In addition, the percentages of cells expressing NFL and MAP2 in five combined treatments are all above 80%. The percentage of cells expressing GFAP is the lowest in the RA treatment group (only 7.7±3.6); however, the percentage of cells expressing GFAP is the highest in the RA+SHH+FGF treatment group (71.1±7.8). Further, the percentage of cells expressing S100 is the lowest in the RA+SHH treatment group. Furthermore, the percentages of cells expressing A2B5, CNPase and O4 in five different combined treatment groups are all above 80%. As to the induction of differentiation of dopaminergic neurons, although the percentage of cells expressing TH is low in the RA treatment group (only 8.7±1.7), the percentage of cells expressing DAT is relatively high in the RA treatment group (about 87.3±6.5). As to the induction of differentiation of cholinergic neurons, although the percentage of cells expressing ChAT in the SHH+FGF treatment group is the lowest, it is still above 80%. As to the induction of differentiation of GABAergic neurons, the percentage of cells expressing GABA in the RA group is the highest (80.5±9.2), and the percentages of cells expressing GABA in the RA+FGF and SHH+FGF groups are lower, about 50%.

While the invention had been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modification and similar structures.

What is claimed is:

1. A cell system for producing neurons via directed differentiation comprising a porcine embryonic stem (pES) cell line derived from pre-implantation blastocytes.

2. The cell system of claim 1, wherein the porcine embryonic stem cell line is the M215-3 strain.

3. A method for inducing neural differentiation of embryonic stem (ES) cells in a two-stage protocol comprising:
(1) culturing ES cells in a suspension culture in ES-cell culture medium (ESM) containing a neurogenic stimulator for a 12-day period;
(2) collecting the cultured cells from the ESM;
(3) plating the collected cells onto gelatin-coated dishes and culturing them for an appropriate period to allow differentiation into dopaminergic neurons, cholinergic (ChAT) neurons or GABAergic (GABA) neurons.

4. The method of claim 3, wherein the neurogenic stimulator is selected from the group consisting of: (1) retinoic acid (RA); (2) sonic hedgehog (SHH); (3) fibroblast growth factor (FGF); and combinations thereof.

5. A method for identifying a neurogenic stimulator comprising the steps of:
(1) culturing the cell system of claim 1 in a suspension culture in ES-cell culture medium (ESM) containing a potential neurogenic stimulator for a 12-day period;
(2) collecting the cultured cells from the ESM;
(3) plating the collected cells onto gelatin-coated dishes and culturing them for an appropriate period to allow differentiation into dopaminergic neurons, cholinergic (ChAT) neurons or GABAergic (GABA) neurons;
(4) detecting biomarkers of interest expressed by pES cell-derived neural cells to determine the effect of the said potential neurogenic stimulator.

6. The method of claim 5, wherein the biomarkers of interest are selected from the group consisting of nestin, neurofilament protein (NFL), microtubule associated protein 2 (MAP2), glial fibrillary acidic protein (GFAP), S100 protein, A2B5, cyclic nucleotide phosphohydrolase (CNPase), O4, tyrosine hydroxylase (TH), dopamine transporter (DAT), choline acetyltransferase (ChAT) and Gamma-aminobutyric acid (GABA).
